(19) United States
(12) Patent Application Publication
(10) Pub. No.: US 2018/0021478 A1
(43) Pub. Date: Jan. 25, 2018

(54) DETOXIFICATION AND STABILIZATION OF IMPLANTABLE OR TRANSPLANTABLE BIOLOGICAL MATERIAL

(71) Applicant: University of the Free State, Bloemfontein (ZA)

(72) Inventors: Dreyer Bester, Bloemfontein (ZA); Francis Edwin Smit, Bloemfontein (ZA); Johannes Van den Heever, Bloemfontein (ZA); Lezelle Botes, Bloemfontein (ZA); Pascal Maria Chris Eugene Dohmen, Berlin (DE)

(21) Appl. No.: 15/545,935

(22) PCT Filed: Jan. 22, 2016
(86) PCT No.: PCT/IB2016/050320

§ 371 Date: Jul. 24, 2017

(30) Foreign Application Priority Data
Jan. 22, 2015 (ZA) 2015/00478

(51) Int. Cl. A61L 27/36; A01N 43/16; A01N 25/02; A01N 43/90

(52) U.S. Cl. CPC A61L 27/3687; A01N 43/90; A01N 43/16; A01N 25/02; A61L 2430/40

(57) ABSTRACT

This invention relates to method of detoxification and stabilization of implantable or transplantable biological material of human or animal origin, the method including the following steps: treatment of the material with an antibiotic solution; treatment of the material in a solution containing an organic acid surfactant bile acid; treatment of the material in a solution to remove the organic acid surfactant bile acid; and treatment of the material in a primary alcohol such as ethanol. The solution containing an organic acid surfactant bile acid contains a synergistic triple combination of the organic acid surfactant bile acid, an anionic surfactant, and a non-ionic surfactant.

### BACKGROUND OF THE INVENTION

Tissue engineering, for example whole organ engineering, could help to address the problems discussed in the background to the invention, since the tissue used is biological and there is no rejection potential. Additionally, there is the potential of tissue regeneration and remodeling. In order to carry out tissue engineering the classical triangle is needed: a scaffold, a large number of different autologous cells and a bioreactor.

U.S. Pat. No. 7,438,850 describes a four-step sterilization method for the production of implantable or transplantable biological material of animal or human origin.

It is an object of this invention to provide an improved method of detoxification and stabilization of implantable or transplantable biological material of human or animal origin.

### SUMMARY OF THE INVENTION

This invention relates to method of detoxification and stabilization of implantable or transplantable biological material of human or animal origin, the method including the following steps:
1) treatment of the material with an antibiotic solution;
2) treatment of the material in a solution containing an organic acid surfactant bile acid (secondary);
3) treatment of the material in a solution to remove the organic acid surfactant bile acid (secondary); and
4) treatment of the material in a primary alcohol such as ethanol; wherein, the solution containing an organic acid surfactant bile acid (secondary) contains:
   the organic acid surfactant bile acid (secondary), preferably deoxycholic acid or a derivative thereof such as ursodeoxycholic acid;
   an anionic surfactant, preferably a sulfate such as sodium dodecyl sulfate, ammonium dodecyl sulfate or potassium lauryl sulfate, and
   a non-ionic surfactant, preferably a polyoxyethylene surfactant such as Triton-x100 or Triton-x200.

The solution containing an organic acid surfactant bile acid (secondary) preferably contains:
the organic acid surfactant bile acid (secondary), preferably deoxycholic acid or a derivative thereof at a concentration of 0.1-2%, typically 0.5% v/v;
the anionic surfactant at a concentration of 1-3%, typically 2% v/v; and
the non-ionic surfactant at a concentration of 0.1-3.0, typically 0.5% v/v.

The antibiotic solution may contain at least one, preferably all of the following antibiotics:
Amphotericin B, in an amount of 5-100, typically 5-15, preferably 10 μg/ml,
Ciprofloxacin, in an amount of 5-200, typically 45-55, preferably 50 μg/ml,
Cefuroxin, in an amount of 20-1500, typically 740-760, preferably 750 μg/ml,
Penicillin, in an amount of 20-1000, typically 180-220, preferably 200 μg/ml,
Streptomycin in an amount of 20-1000, typically 180-220, typically 200 μg/ml.

Preferably, the antibiotic solution contains no Ca or Mg.

The solution to remove the organic acid surfactant bile acid (secondary) contains a lipopeptide anticmicrobial agent such as Fengycin or Iturin A in an amount of 5-800, preferably 20 μg/ml.

After step 4), the material is introduced to a storage solution containing antibiotics, preferably selected from one or all of the following antibiotics:
Amphotericin B, typically in an amount of 5-15, preferably 10 μg/ml,
Penicillin, typically in an amount of 80-120, typically 100 μg/ml,
Streptomycin, typically in an amount of 80-120, typically 100 μg/ml.

The invention also relates to the solution containing an organic acid surfactant bile acid (secondary) for use in a method of detoxification and stabilization of implantable or transplantable biological material of human or animal origin, said solution containing:
the organic acid surfactant bile acid (secondary), preferably deoxycholic acid or a derivative thereof such as ursodeoxycholic acid;
an anionic surfactant, preferably a sulfate such as sodium dodecyl sulfate, ammonium dodecyl sulfate or potassium lauryl sulfate, and
a non-ionic surfactant, preferably a polyoxyethylene surfactant such as Triton-x100 or Triton-x200.

### BRIEF DESCRIPTION OF THE DRAWINGS

FIG. 1 is a light microscopy photograph of tissue from a heart valve wall treated with a deoxycholic acid solution;

FIG. 2 is a light microscopy photograph of tissue from a heart valve wall treated with a deoxycholic acid and sodium dodecyl sulfate solution;

FIG. 3 is a light microscopy photograph of tissue from a heart valve wall treated with a deoxycholic acid, sodium dodecyl sulfate and Triton-x100 solution of the invention;

FIG. 4 is a light microscopy photograph of tissue from a leaflet of a heart valve wall treated with a deoxycholic acid solution;

FIG. 5 is a light microscopy photograph of tissue from a leaflet of a heart valve wall treated with a deoxycholic acid and sodium dodecyl sulfate solution;

FIG. 6 is a light microscopy photograph of tissue from a leaflet of a heart valve wall treated with a deoxycholic acid, sodium dodecyl sulfate and Triton-x100 solution of the invention;

FIG. 7 is a light microscopy photograph of pericardial tissue treated with a deoxycholic acid solution;

FIG. 8 is a light microscopy photograph of pericardial tissue treated with a deoxycholic acid and sodium dodecyl sulfate solution;

FIG. 9 is a light microscopy photograph of pericardial tissue treated with a deoxycholic acid, sodium dodecyl sulfate and Triton-x100 solution of the invention; and

FIG. 10 is a light microscopy photograph of myocardium tissue treated with a deoxycholic acid, sodium dodecyl sulfate and Triton-x100 solution of the invention.

### DESCRIPTION OF PREFERRED EMBODIMENTS

In accordance with the method of the present invention, tissue of human or animal origin is treated in four successive steps:
1) treatment of the tissue with an antibiotic solution;
2) treatment of the tissue in a solution containing an organic acid surfactant bile acid (secondary);
3) treatment of the tissue in a solution to remove the organic acid surfactant bile acid (secondary); and
4) treatment of the tissue in a primary alcohol.

The solution of Step 2) is a physiological solution containing an organic acid surfactant bile acid (secondary), contains a triple combination of:
deoxycholic acid or derivative thereof,
an anionic surfactant, preferably a sulfate such as sodium dodecyl sulfate, ammonium dodecyl sulfate or potassium lauryl sulfate, and
a non-ionic surfactant.

Deoxycholic acid (DOA), is a bile acid (secondary) an organic surfactant. Alternatively a derivative thereof such as ursodeoxycholic acid can also be used. It also takes care of the lipid parts of the membrane, not only the protein part. Additionally it has an anti-inflammatory activity which is also important in the production of an extracellular matrix.

The preferred anionic surfactant is sodium dodecyl sulfate (SDS), alternative anionic surfactants are ammonium dodecyl sulfate and potassium lauryl sulfate, which are of the same group, however a little different. These are anionic surfactant (anorganic) or detergent surfactant which denaturate proteins, but also microbicide including enveloped and non-enveloped viruses will be destroyed.

The preferred non-ionic surfactant is Triton-x100 (a polyoxyethylene surfactant) which will not denaturate proteins but results in membrane distortion to prepare the tissue for sodium dodecyl sulfate and deoxycholic acid to destroy the tissue (denaturate).

This triple combination has a synergistic effect: i.e. the combination of all three components allows the concentration of the individual components to be reduced. A lower concentration of components means that, in use, there is less chance that the stability of the extracellular matrix/scaffold will be changed during the detoxification and stabilization of the scaffold.

After step 4), the tissue is rinsed to remove all debridement out of the extracellular matrix. Thereafter, the material is introduced to a storage solution containing antibiotics, preferably selected from one or all of the following antibiotics:
Amphotericin B in an amount of 5-15 μg/ml,
Penicillin in an amount of 80-120 μg/ml,
Streptomycin in an amount of 80-120 μg/ml.

The method may be used in the preparation of a heart, heart valves, grafts, patch material etc. and also other organs or tissue such as omentum.

The invention is described in more detail with reference to the following Examples. The invention is not restricted to these Examples.

### Examples

An Example of the invention is the preparation of a large size heart, which was decellularized to create a scaffold on which autologous cells can be transplanted and later on implanted.

Technique to modify tissue by detoxification and stabilization:

Step 1)—treatment of the tissue with an antibiotic solution

The antibiotic solution without Ca or Mg contains a cocktail of antibiotics and antimycotic medication with flow or without flow at a shaker for several hours and at room temperature or at 37° C.

| | |
|---|---|
| 10 μg/ml | Amphotericin B |
| 50 μg/ml | Ciprofloxacin |
| 750 μg/ml | Cefuroxin |
| 200 U/ml | Penicillin |
| 200 μg/ml | Streptomycin |

Thereafter the tissue is treated with distilled or purified water also for a particular time 15 minutes to 1 hour at room temperature or 37° C.

Step 2)—treatment of the tissue in a solution containing an organic acid surfactant bile acid (secondary)

The tissue is treated with a combination of deoxycholic acid (DOA) (0.5% v/v), sodium dodecyl sulfate (SDS) (2% v/v), and Triton x-100 (0.5% v/v). These substances have been used in the past, however never all together since there is a synergic effect in case using them together. Therefore the concentration can be lower and there is less chance that the stability of the extracellular matrix/scaffold can be changed. The collagen can be destroyed and deterioration can be increased due to this. This step is carried out at a particular temperature (room or 37° C.) for several hours or days (long).

Step 3)—treatment of the tissue in a solution to remove the organic acid surfactant bile acid (secondary)

The tissue is treated with Fengycin 100 μg/ml in DMSO 5 mmol to sterilize and to remove the DOA/SDS and Triton out of the tissue. It is also possible to use Iturin A for several hours. These are lipopeptide antimicrobial agents that are also antifungal. It will stabilize the tissue more. Concentrations can be changed, depending on the time. Temperature can also be different (room temperature or 37° C. is optimal). A shaker or flow can be used.

Step 4)—treatment of the tissue in a primary alcohol

Ethanol or another alcohol should be used to stabilize the tissue but will also sterilize the tissue. Again with or without shaker or under flow conditions for different time (several hours and at different temperature (room temperature or 37° C. is optimal).

Extensive rising of the tissue to get all the debridement out of the extracellular matrix. It would also be possible to control this by measurement to minimize the debridement at a minimum on the end.

Final step is storage with a specific store solution:

| | |
|---|---|
| 10 μg/ml | Amphotericin B |
| 100 μg/ml | Penicillin (reduced concentration) |
| 100 μg/ml | Streptomycin (reduced concentration) |

The process of the invention described above was carried out on different tissues and comparative tests were conducted using a single solution containing either deoxycholic acid, or sodium dodecyl sulfate, or Triton-X100, and a double solution containing either deoxycholic acid with sodium dodecyl sulfate, or deoxycholic acid with Triton-X100, or sodium dodecyl sulfate with Triton-X100. Only in the triple deoxycholic acid solution of the invention containing an organic acid surfactant bile acid (secondary); an anionic surfactant, and a non-ionic surfactant results in a cell free tissue without destroying the extracellular structures.

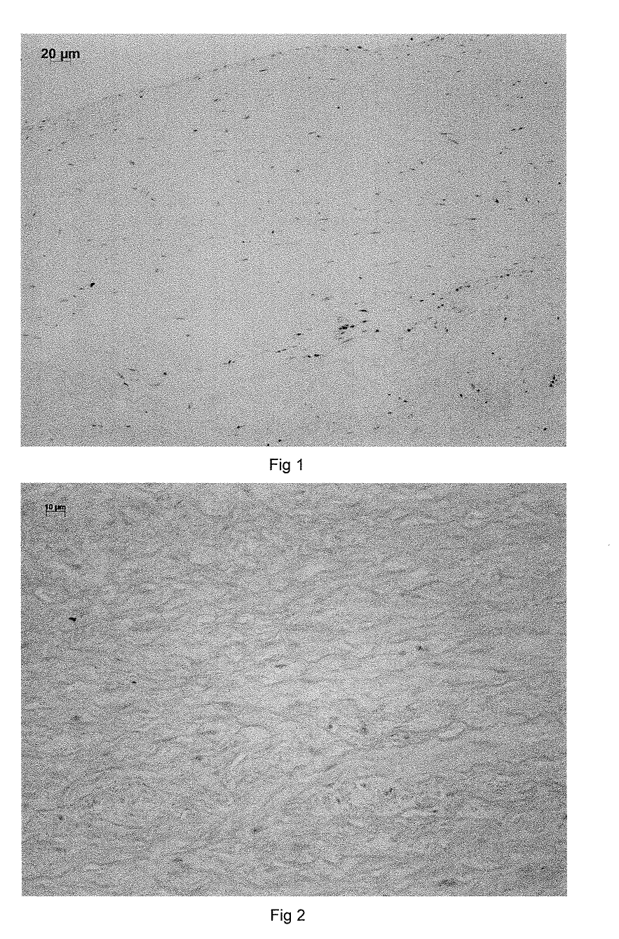

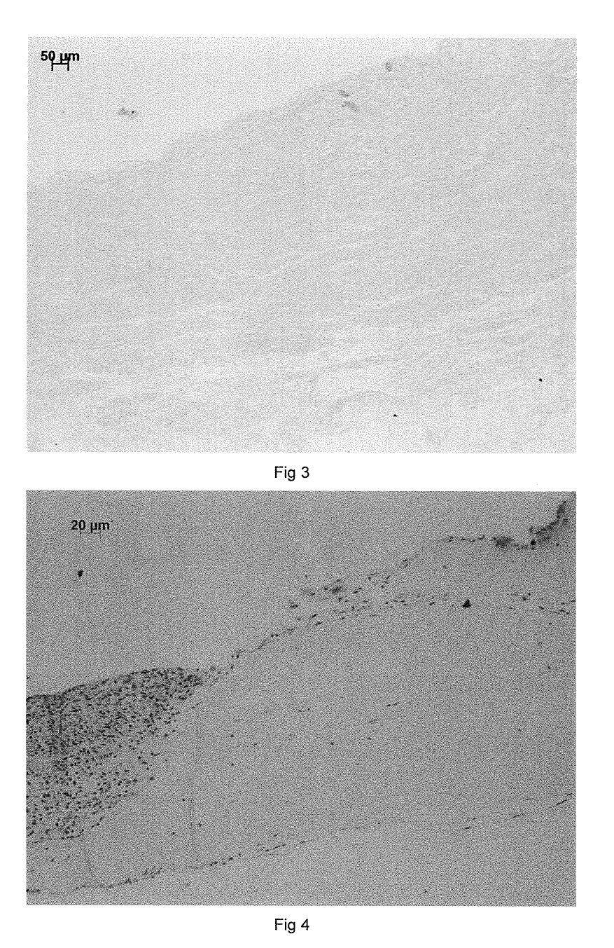

FIG. 1 is a light microscopy photograph of tissue from a heart valve wall treated with the single deoxycholic acid solution. FIG. 2 is a light microscopy photograph of tissue from a heart valve wall treated with the double deoxycholic acid solution. FIG. 3 is a light microscopy photograph of tissue from a heart valve wall treated with the triple deoxycholic acid solution of the invention. From FIG. 1, it can be seen that treatment with a single solution containing deoxycholic acid, one a large part of the tissue is free of cells, however not completely. FIG. 2, which shows the result of treatment with the double deoxycholic acid solution shows an improvement, with additional reduction of cells in the tissue. However, as shown in FIG. 3, it is only the triple deoxycholic acid solution of the invention where there are no cells available any longer, and thus achieves complete decellularization.

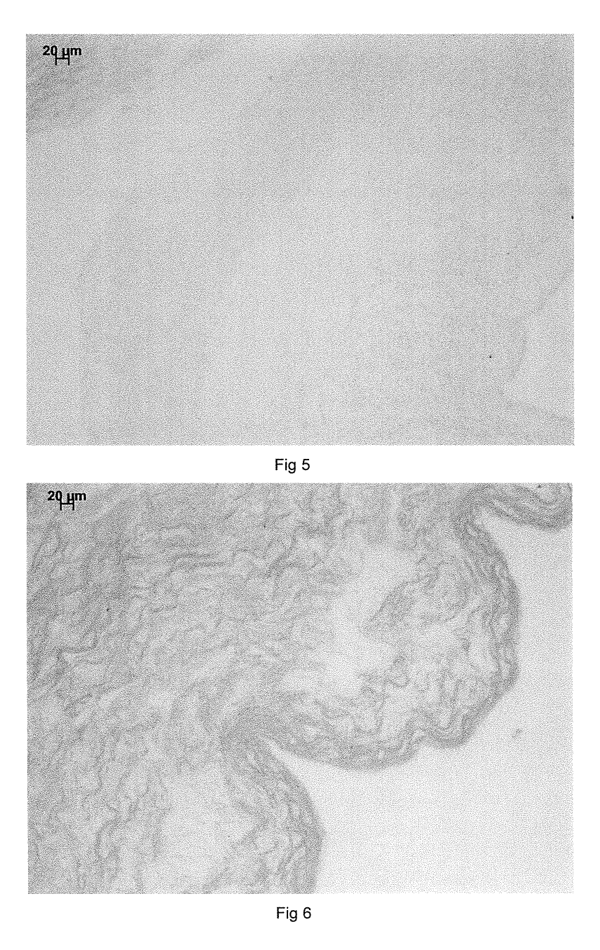

FIG. 4 is a light microscopy photograph of tissue from a leaflet of a heart valve wall treated with the single deoxycholic acid solution. FIG. 5 is a light microscopy photograph of tissue from a leaflet of a heart valve wall treated with the double deoxycholic acid solution. FIG. 6 is a light microscopy photograph of tissue from a leaflet of a heart valve wall treated with the triple deoxycholic acid solution of the invention. From FIG. 4, it can be seen that treatment with a single solution containing deoxycholic acid, one a large part of the tissue is free of cells, however not completely. FIG. 5, which shows the result of treatment with the double deoxycholic acid solution shows an improvement, with additional reduction of cells in the tissue. However, as shown in FIG. 6, it is only the triple deoxycholic acid solution of the invention where there are no cells available any longer, and thus achieves complete decellularization.

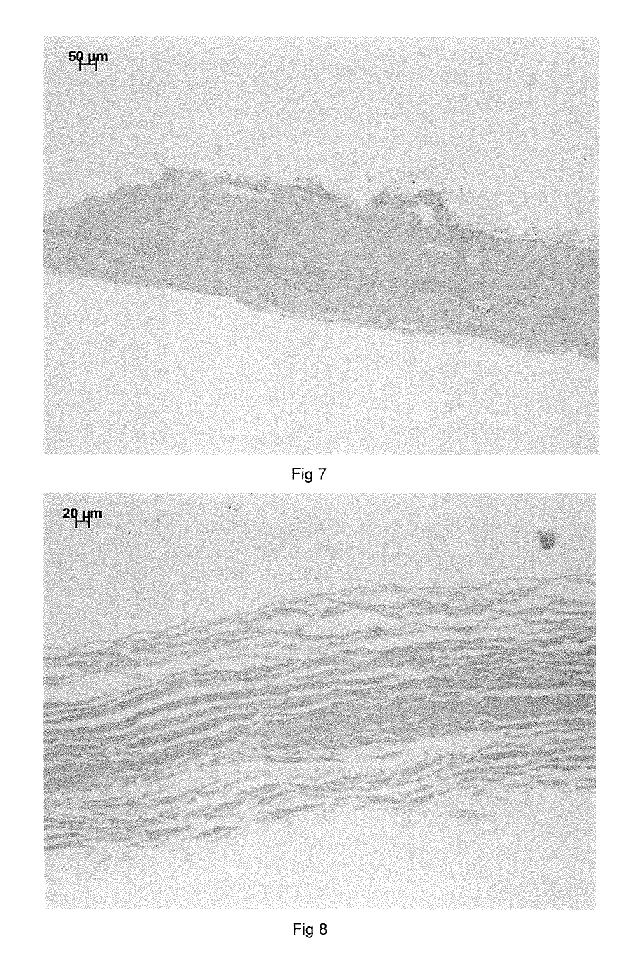

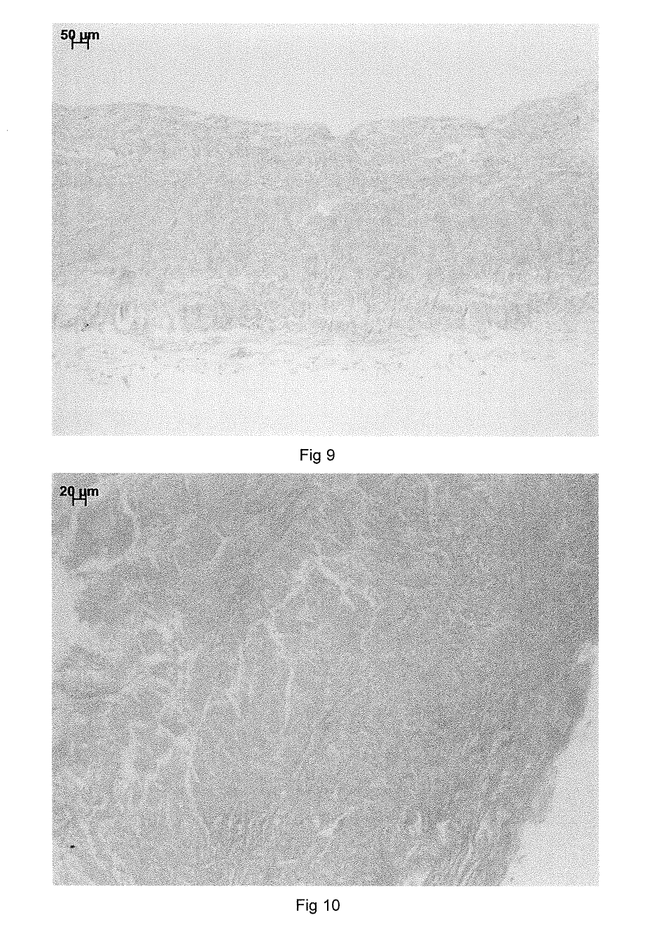

FIG. 7 is a light microscopy photograph of pericardial tissue treated with the single deoxycholic acid solution. FIG. 8 is a light microscopy photograph of pericardial tissue treated with the double deoxycholic acid solution. FIG. 9 is a light microscopy photograph of pericardial tissue treated with the triple deoxycholic acid solution of the invention. From FIG. 7, it can be seen that treatment with a single solution containing deoxycholic acid, one a large part of the tissue is free of cells, however not completely. FIG. 8, which shows the result of treatment with the double deoxycholic acid solution shows an improvement, with additional reduction of cells in the tissue. However, as shown in FIG. 9, it is only the triple deoxycholic acid solution of the invention where there are no cells available any longer, and thus achieves complete decellularization.

FIG. 10 is a light microscopy photograph of myocardium tissue treated with the triple deoxycholic acid solution of the invention. As shown in FIG. 10, the triple deoxycholic acid solution of the invention, and thus achieves complete decellularization.

1. A method of detoxification and stabilization of implantable or transplantable biological material of human or animal origin, the method including the following steps:
1) treatment of the material with an antibiotic solution;
2) treatment of the material in a solution containing an organic acid surfactant bile acid (secondary);
3) treatment of the material in a solution to remove the organic acid surfactant bile acid (secondary); and
4) treatment of the material in a primary alcohol such as ethanol;
wherein, the solution at Step 2) containing an organic acid surfactant bile acid (secondary) contains:
the organic acid surfactant bile acid (secondary);
an anionic surfactant, and
a non-ionic surfactant.

2. The method claimed in claim 1, wherein the organic acid surfactant bile acid (secondary) is deoxycholic acid or a derivative thereof.

3. (canceled)

4. The method claimed in claim 2, wherein the anionic surfactant is a sulfate.

5. The method claimed in claim 4, wherein the sulfate is sodium dodecyl sulfate, ammonium dodecyl sulfate or potassium lauryl sulfate.

6-8. (canceled)

9. The method claimed in claim 1, wherein the ion solution containing an organic acid surfactant bile acid (secondary) contains:
the organic acid surfactant bile acid (secondary) at a concentration of 0.5% v/v;
the anionic surfactant at a concentration of 2% v/v; and
the Triton-x100 surfactant at a concentration 0.5% v/v.

10. The method claimed in claim 1, wherein the antibiotic solution contains at least one of the following antibiotics:
Amphotericin B,
Ciprofloxacin,
Cefuroxin,
Penicillin.

11. The method claimed in claim 10, wherein the antibiotic solution contains the following antibiotics:
Amphotericin B, in an amount of 5-100 μg/ml,
Ciprofloxacin, in an amount of 5-200 μg/ml,
Cefuroxin, in an amount of 20-1500 μg/ml,
Penicillin, typically in an amount of 20-1000 μg/ml,
Strepotomycin, in an amount of 20-1000 μg/ml.

12. The method claimed in claim 11, wherein the antibiotic solution contains the following antibiotics:
Amphotericin B, in an amount of 5-15 μg/ml,
Ciprofloxacin, in an amount of 45-55 μg/ml,
Cefuroxin, in an amount of 740-760 μg/ml,
Penicillin, typically in an amount of 180-220 μg/ml,
Streptomycin in an amount of 180-220 μg/ml.

13. The method claimed in claim 12, wherein the antibiotic solution contains the following antibiotics:
Amphotericin B, in an amount of 10 μg/ml,
Ciprofloxacin, in an amount of 50 μg/ml,
Cefuroxin, in an amount of 750 μg/ml,
Penicillin, in an amount of 200 μg/ml,
Streptomycin, in an amount of 200 μg/ml.

14. The method claimed in claim 1, wherein the antibiotic solution contains no Ca or Mg.

15. The method claimed in claim 1, wherein the solution to remove the deoxycholic acid or a derivative thereof contains a lipopeptide anticmicrobial agent.

16. The method claimed in claim 15, wherein the lipopeptide anticmicrobial agent is Fengycin or Iturin A.

17. The method claimed in claim 1 wherein, after step 4), the material is introduced to a storage solution containing antibiotic/s.

18. The method claimed in claim 17, wherein the storage solution contains one or all of the following antibiotics:
Amphotericin B,
Penicillin,
Streptomycin.

19. The method claimed in claim 18, wherein the storage solution contains the following antibiotics:
Amphotericin B, in an amount of 5-15 μg/ml,
Penicillin, in an amount of 80-120 μg/ml,
Streptomycin, in an amount of 80-120 μg/ml.

20. The method claimed in claim 19, wherein the storage solution contains the following antibiotics:
Amphotericin B, in an amount of 10 μg/ml,
Penicillin, in an amount of 100 μg/ml,
Streptomycin, in an amount of 100 μg/ml.

21. A solution containing an organic acid surfactant bile acid (secondary) for use in a method of detoxification and stabilization of implantable or transplantable biological material of human or animal origin, the solution containing:
the organic acid surfactant bile acid (secondary) at a concentration of 0.1-2% v/v;
an anionic surfactant at a concentration of 1-3% v/v, and
Triton-x100 surfactant at a concentration of 0.1-3% v/v.

22. The solution claimed in claim 21, the organic acid surfactant bile acid (secondary) is deoxycholic acid or ursodeoxycholic acid.

23. (canceled)

24. The solution claimed claim 21, wherein the anionic surfactant is a sulfate.

25. The solution claimed in claim 24, wherein the sulfate is sodium dodecyl sulfate, ammonium dodecyl sulfate or potassium lauryl sulfate.

26-28. (canceled)

29. The solution claimed in claim 21, wherein the solution containing an organic acid surfactant bile acid (secondary) contains:
the organic acid surfactant bile acid (secondary) at a concentration of 0.5% v/v;
the anionic surfactant at a concentration of 2% v/v; and
the Triton-x100 surfactant at a concentration 0.5% v/v.
